(19) United States
(12) Patent Application Publication
(10) Pub. No.: US 2007/0267018 A1
(43) Pub. Date: Nov. 22, 2007

(54) ENHANCED CONVECTION HEAT-TREATMENT SYSTEM AND METHOD

(75) Inventor: Hans Paller, Dallas, TX (US)

Correspondence Address: DAVIS WRIGHT TREMAINE, LLP, 1201 Third Avenue, Suite 2200, SEATTLE, WA, 98101-3045, US

(73) Assignee: Lang Manufacturing Company, Everett, WA (US)

(21) Appl. No.: 11/419,476

(22) Filed: May 19, 2006

(51) Int. Cl. A61M 16/00

(52) U.S. Cl. 128/204.23

(57) ABSTRACT

An enhanced convection heat-treatment system and method is configured to direct airflow with a plurality of air-paths past product, such as loaves of bread, to impart heat to the product and past a heating system to impart heat to the air. The enhanced system operates in a first mode with airflow along each of the plurality of air-paths in a first direction. The enhanced system operates in a second mode with airflow reversed although substantially along each of the same plurality of air-paths in an opposite second direction to assist in uniform heat-treatment of the product. For instance, as a convection oven, the enhanced convection heat-treatment system could distribute airflow evenly over a baked product resulting in an even bake of the product.

### BACKGROUND OF THE INVENTION

1. Field of the Invention

The present invention is related to convection heat-treatment systems such as convection ovens.

2. Description of the Related Art

Conventional convection heat-treatment systems, such as convection ovens, generally include a product treatment area, an air-heating system, and an airflow system. The product treatment area generally includes multiple racks that support product, such as food including baked goods and roasted proteins, at various vertical elevations within the product treatment area to be baked, roasted, otherwise cooked, and/or otherwise heat-treated. The air-heating system can include one or more fuel-fed burners or electric elements. The airflow system generally passes air heated by the air-heating system through the product treatment area, by the product, and back to the air-heating system to be reheated. Convection heat-treatment systems specifically for baking or roasting also typically use a steam generator to introduce steam into the product treatment area for predefined periods of time (usually of short duration) to impart a desired appearance or texture to product located in the product area.

With convection heat-treatment systems, it is often desirous to uniformly heat-treat product in the product treatment area. For instance, uniformly heat-treated baked goods, such as bread, may be more desirable if they have an even finished surface appearance than baked goods having variation in appearance such as with color or texture. Unfortunately, conventional attempts for uniform convection heat-treatment involve elaborate mechanical or manual approaches.

For instance, mechanical approaches for uniform convection heat-treatment include complicated automated rack rotation systems. In the absence of automation, other conventional approaches for uniform convection heat-treatment of product include manual removal and rotation of product during heat-treatment. These and other elaborate conventional approaches to uniform convection heat-treatment of product can have detrimental consequences including increased production cost of product, increased manufacturing cost of convection heat-treatment systems, decreased ease-of-use, decreased operational reliability, and less than hoped-for results.

### BRIEF DESCRIPTION OF THE SEVERAL VIEWS OF THE DRAWING(S)

FIG. 1 is an elevational front view of an enhanced convection heat-treatment system.

FIG. 2 is a top sectional view of the enhanced convection heat treatment system taken along the 2-2 line of FIG. 1 showing airflow in a first direction.

FIG. 3 is a top sectional view of the enhanced convection heat treatment system taken along the 3-3 line of FIG. 1 showing airflow in a second direction.

FIG. 4 is an elevational front view of the enhanced convection heat-treatment system of FIG. 1 shown containing product.

FIG. 5 is a top fragmentary view of the enhanced convection heat-treatment system shown containing product.

FIG. 6 is an elevational sectional view of the enhanced convection heat-treatment system taken along the 6-6 line of FIG. 5 showing airflow in a first direction.

FIG. 7 is an elevational sectional view of the enhanced convection heat-treatment system taken along the 7-7 line of FIG. 5 showing airflow in a first direction.

FIG. 8 is an elevational sectional view of the enhanced convection heat-treatment system taken along the 8-8 line of FIG. 5 showing airflow in a first direction.

FIG. 9 is an elevational sectional view of the enhanced convection heat-treatment system taken along the 9-9 line of FIG. 5 showing airflow in a first direction.

FIG. 10 is an elevational sectional view of the enhanced convection heat-treatment system taken along the 10-10 line of FIG. 5 showing airflow in a second direction.

FIG. 11 is an elevational sectional view of the enhanced convection heat-treatment system taken along the 11-11 line of FIG. 5 showing airflow in a second direction.

FIG. 12 is an elevational sectional view of the enhanced convection heat-treatment system taken along the 12-12 line of FIG. 5 showing airflow in a second direction.

FIG. 13 is an elevational sectional view of the enhanced convection heat-treatment system taken along the 13-13 line of FIG. 5 showing airflow in a second direction.

FIG. 14 is an exploded perspective view of a louver assembly and shroud of the enhanced convection heat-treatment system of FIG. 2 as unassembled.

FIG. 15 is a perspective view of the louver assembly and shroud of FIG. 14 as assembled.

### DETAILED DESCRIPTION OF THE INVENTION

As discussed herein, an enhanced convection heat-treatment system and method is configured to direct airflow with a plurality of air-paths past product, such as loaves of bread, to impart heat to the product and past a heating system to impart heat to the air. The enhanced system operates in a first mode with airflow along each of the plurality of air-paths in a first direction. The enhanced system operates in a second mode with airflow reversed although substantially along each of the same plurality of air-paths in an opposite second direction to assist in uniform heat-treatment of the product. For instance, as a convection oven, the enhanced convection heat-treatment system could distribute airflow evenly over a baked product resulting in an even bake of the product.

The enhanced system includes an air treatment area and a product treatment area. The air treatment area has a heating system that imparts heat to air and a steam system that imparts steam to air. The air treatment area is bounded by an intermediate panel such as a shroud. The product treatment area is bounded by an air distribution panel such as a louver assembly that directs air passing through it to maintain substantially consistent airflow through the louver assembly regardless of vertical elevation. A bi-directional axial propeller type fan is co-located with the shroud adjacent the air treatment area and on a side of the louver assembly opposite the product treatment area. In implementations, airflow rate from the fan in a first mode is substantially similar to airflow rate from the fan in a second mode although second mode airflow is substantially opposite in direction from first mode airflow. Side passageways extend between the product treatment area and the air treatment area.

When operating in the first mode, the fan blows air in a first direction to increase air pressure in the air treatment area behind the shroud relative to air pressure found in the product area. Having a higher pressure than air in the product treatment area, air in the air treatment area passes through side passageways at a substantially consistent rate regardless of vertical elevation into the product treatment area where product is arranged in vertically stacked racks. In the first mode, the fan intakes air from the product treatment area through the louver assembly, which assists in maintaining substantially consistent intake of air from the product treatment area into the fan regardless of vertical elevation of the air in the product treatment area.

When operating in a second mode the fan moves treated air in a second direction from the air treatment area toward the louvers into the product treatment area at a substantially consist rate regardless of elevation due to action by the louvers. Consequently, air pressure in the product treatment area is increased relative to air pressure found in the air treatment area. In the second mode, having a higher pressure than air in the air treatment area, air in the product treatment area passes through the side passageways at a substantially consistent rate regardless of vertical elevation into the air treatment area to be re-treated.

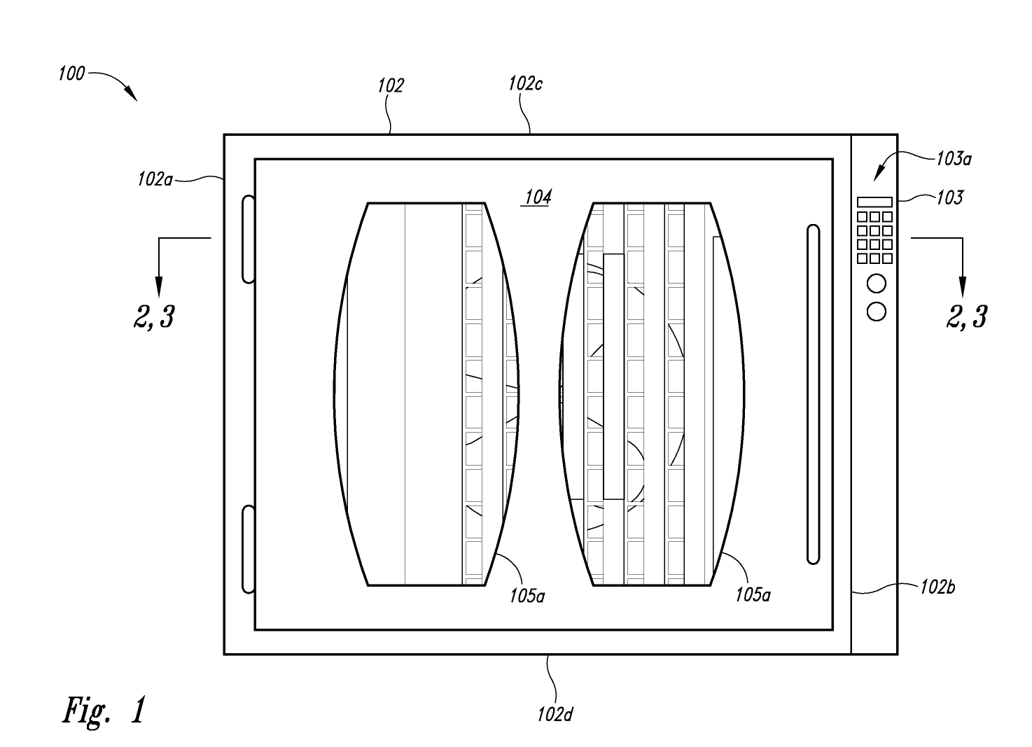

A convection oven implementation 100 of an enhanced convection heat-treatment system is shown in FIG. 1 as having an oven housing 102 with a left sidewall 102a, a right sidewall 102b, a top wall 102c, and a bottom wall 102d. The convection oven 100 further includes an external enclosure 103 containing electronics (not shown) electrically coupled to a control panel 103a. The convection oven 100 has a front door 104 for accessing the interior of the oven housing 102. The front door 104 includes observation windows 105a for viewing product inside the convection oven 100.

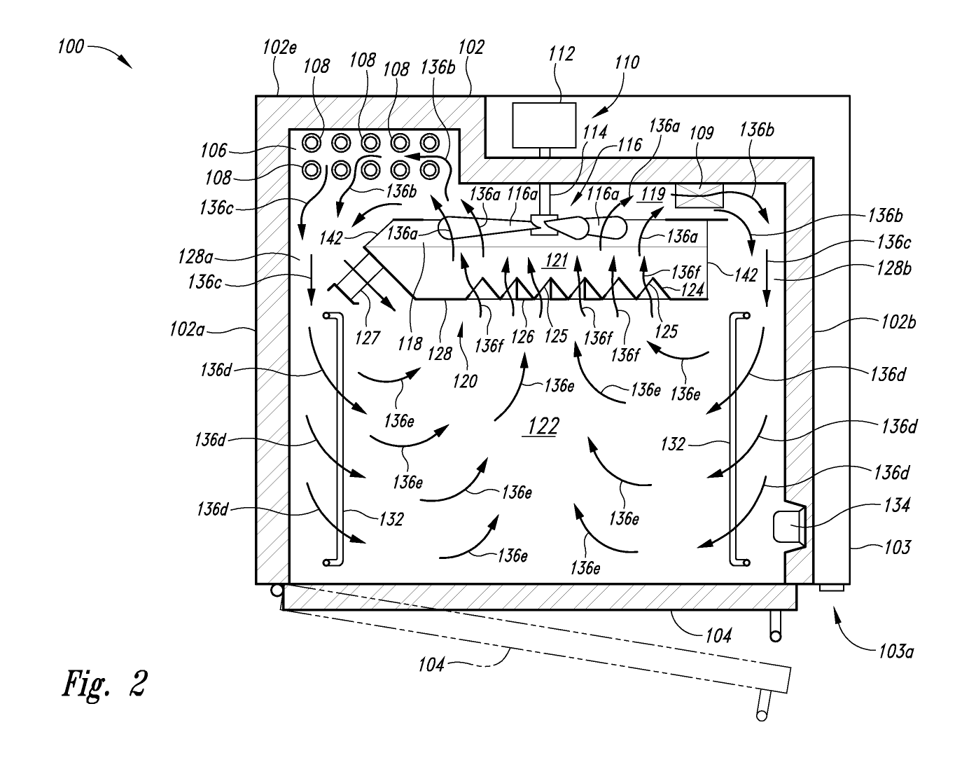

As shown in FIG. 2, the oven housing 102 further includes a rear wall 102e. Mounted adjacent the rear wall 102e is a heat exchanger 106 including exchanger tubes 108 as part of a heating system for imparting heat to air passing the exchanger tubes. In other implementations, other types of heating systems can be used, such as those using resistive heating elements, so long as heat is somehow imparted to air. A steam system 109 is also located adjacent the rear wall 102e. The steam system 109 can take the form of a thermal mass, such as an iron mass that collects heat, and an associated source of water that delivers water onto the thermal mass where the heat of the thermal mass converts the liquid water into steam. Other implementations can use other configurations for the steam system 109. The convection oven 100 has an axial propeller type fan 110 with a motor 112 located in the external enclosure 103 rearward of the rear wall 102e and outside of the oven housing 102. The motor 112 is coupled to a shaft 114 that passes from the external enclosure 103 through the rear wall 102e inside the oven housing 102 where a propeller 116 having blades 116a is positioned and coupled to the shaft. The propeller 116 is encompassed by an intermediate panel such as a shroud 118 that also serves to partition off an air treatment area 119 between the shroud and the rear wall 102e wherein the exchanger tubes 108 and the steam system 109 are located.

Spaced forward of the propeller 116 and the shroud 118 is an air distribution panel such as a louver assembly 120 with a staging area 121 bounded therebetween. A product treatment area 122 is located between the louver assembly 120 and the front door 104. The louver assembly 120 has vertical slats 124, openings 125, baffles 126, and other structural members, further discussed below, to maintain substantially consistent airflow regardless of vertical elevation for airflow approaching the louver assembly from the product treatment area 122 or for airflow departing from the louver assembly into the product treatment area. The louver assembly 120 further includes bracing 127 and a frame member 128 to structurally secure the louver assembly.

As shown, the shroud 118 and the louver assembly 120 do not extend fully to the left sidewall 102a or the right sidewall 102b. Consequently, extending from the air treatment area 119 to the product treatment area 122 along the left sidewall 102a and the right sidewall 102b are a left air passageway 128a and a right air passageway 128b, respectively.

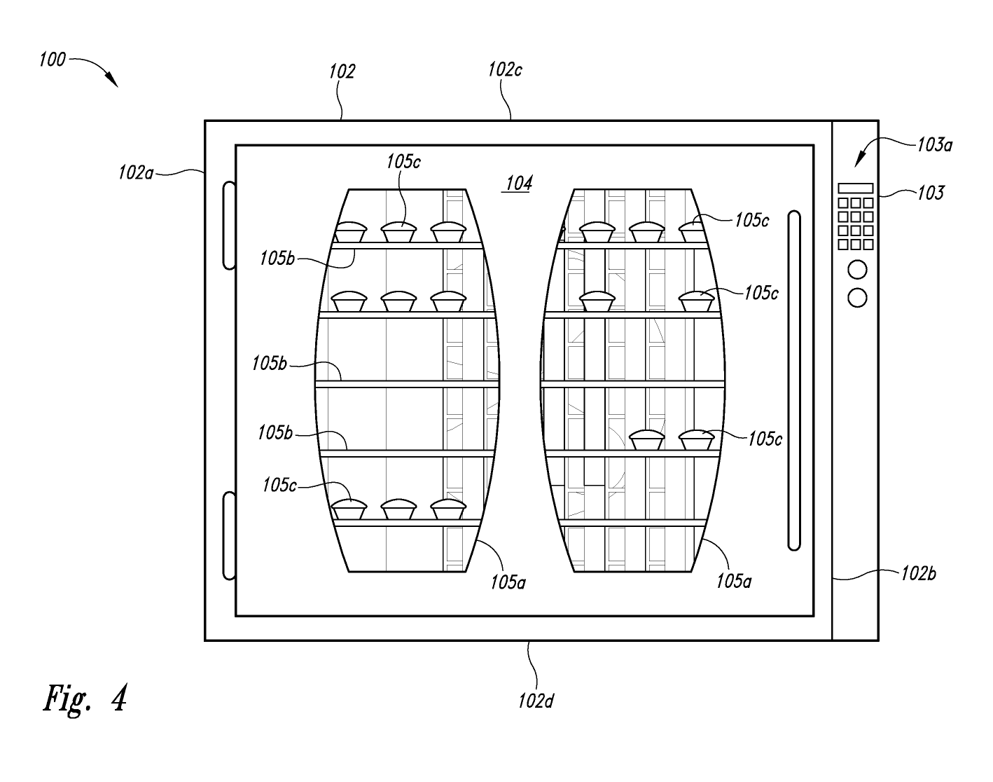

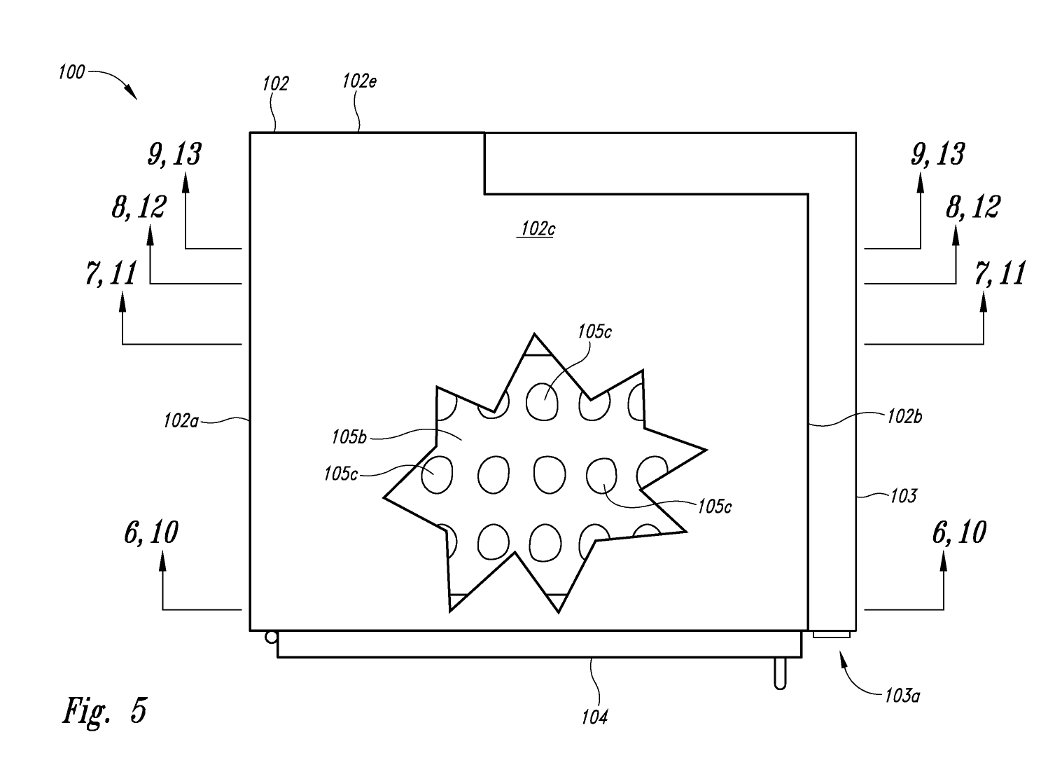

Located in the product treatment area 122 near the left sidewall 102a and the right sidewall 102b are left and right side rack supports 132 to receive racks 105b (shown in FIG. 4) to hold product, such as bread 105c (shown in FIG. 4 and FIG. 5), to be heat-treated in the product treatment area 122. Vertical position of the rack supports 132 may be adjusted to accommodate the height of the product being heat-treated. An interior light 134 (shown in FIG. 2) assists in viewing of product in the product treatment area 122 through the observation windows 105a of the front door 104.

As shown in FIG. 2, the fan 110 operates in a first mode to move air as airflow 136a in a first direction into the air treatment area 119 to increase air pressure in the air treatment area behind the shroud 118 relative to air pressure found in the product treatment area 122. Air in the air treatment area 119 passes around the exchanger tubes 108 as airflow 136b to obtain heat and the steam system 109 to obtain steam. Having a higher pressure than air in the product treatment area 122, the air in the air treatment area 119 leaves the air treatment area as airflow 136c.

The airflow 136c passes through the left air passageway 128a and the right air passageway 128b as substantially vertically undifferentiated, having a substantially consistent rate and direction regardless of vertical elevation within the convection oven 100. Air enters from the left air passageway 128a and from the right air passageway 128b into the product treatment area 122 as airflow 136d and moves as airflow 136e past product 105c, which is arranged on vertically stacked racks 105b (shown in FIG. 4).

Since the airflow 136c passes through the left air passageway 128a and the right air passageway 128b as substantially vertically undifferentiated, the airflow 136d enters into the product treatment area 122 also to move past product as airflow 136e as substantially vertically undifferentiated. In the first mode, the fan 110 intakes air from the staging area 121 resulting in an air pressure lower in the staging area than found in the product treatment area 122. Due to the pressure differences and also construction of the louver assembly 120, air in the product treatment area 122 passes through the louver assembly 120 as airflow 136f in a substantially vertically undifferentiated manner into the staging area 121.

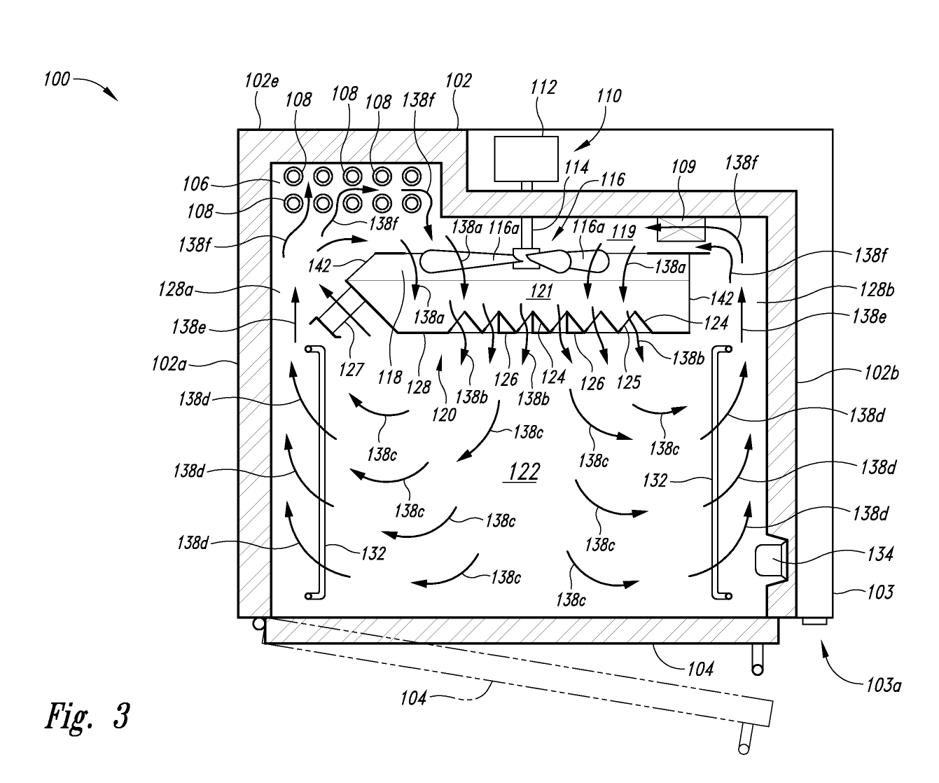

As shown FIG. 3, the fan 110 operates in a second mode to move air in a second direction, opposite from the first direction, from the air treatment area 119 into the staging area 121 as airflow 138a to increase air pressure in the staging area relative to air pressure in the product treatment area 122. Airflow direction is changed from the first direction to the second direction by reversing the direction of rotation of the propeller 116. In a typical operation, the fan 110 is changed from operating between one of the first mode or the second mode to the other of the first mode or the second mode at some point in the heat treatment of the product. Changing between the first mode and the second mode of operation of the fan 110 could also be done more than once in the heat treatment of the product depending upon the characteristics of the particular product being heat-treated. Due to the pressure differences and construction of the louver assembly 120, air from the staging area 119 flows through the louver assembly 120 and into the product treatment area 122 as airflow 138b. Air as airflow 138c then circulates around the product 105c in a substantially similar manner as the airflow 136e as to being substantially vertically undifferentiated and traveling substantially similar pathways as the airflow 136e except that the airflow 138c travels in a direction reverse of the airflow 136e.

In some implementations, the propeller 116 moves air at the same volume or rate in either of the first mode of the fan 110 or the second mode of the fan. Such an implementation of the propeller 116 could have the blades 116a of the propeller 116 being flat and possibly including orientation at an approximate 45° pitch. Other implementations may use versions of the propeller 116 that move air at different rates depending upon the direction that the propeller is spinning to accommodate for differences in how airflow is managed in the convection oven 100 with an object in whatever implementations to have the airflow 138c traveling substantially similar pathways as the airflow 136e through the product treatment area 122 except that the airflow 138c travels in a direction reverse of the airflow 136e direction.

As air is being moved into the product treatment area 122 from the air treatment area 119 by the fan 110, air pressure is increased in the product treatment area compared with the air treatment area. Due to this pressure difference, air from the product treatment area 122 leaves the product treatment area as airflow 138d and flows through the left air passageway 128a and the right air passageway 128b as airflow 138e to enter into the air treatment area 119. Air then circulates as airflow 138f in the air treatment area 119 to pass by the exchanger tubes 108 to obtain heat and to pass by the steam system 109 to obtain steam.

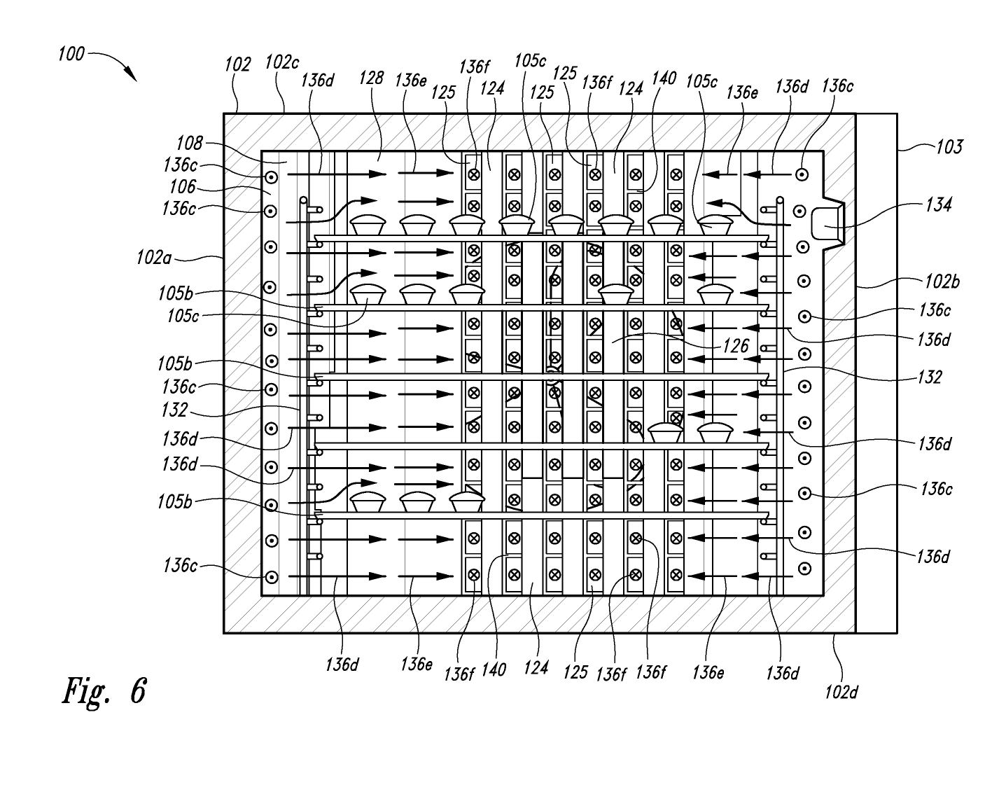

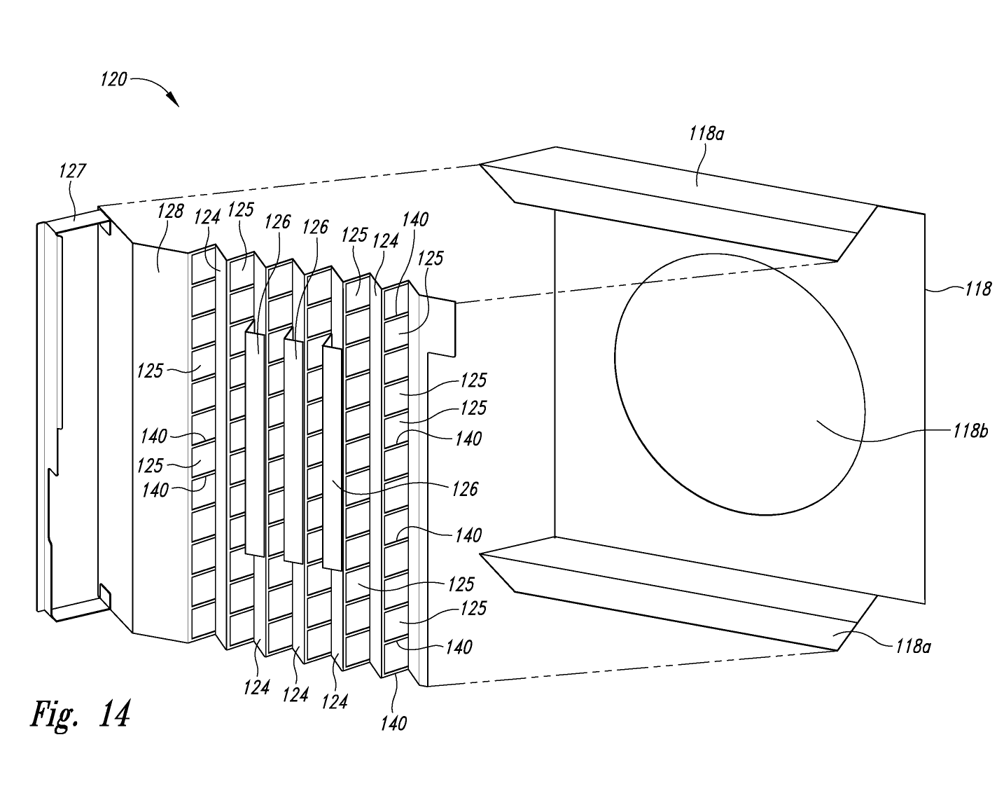

Nomenclature for FIGS. 6-13 include a “.” inside a circle indicating airflow out of the figure sheet and an “x” inside a circle indicating airflow into the figure sheet. As shown in FIG. 6 and better shown in FIG. 14, the louver assembly 120 has horizontal members 140, which support the vertical slats 124 and help to maintain substantially vertical undifferentiated airflow.

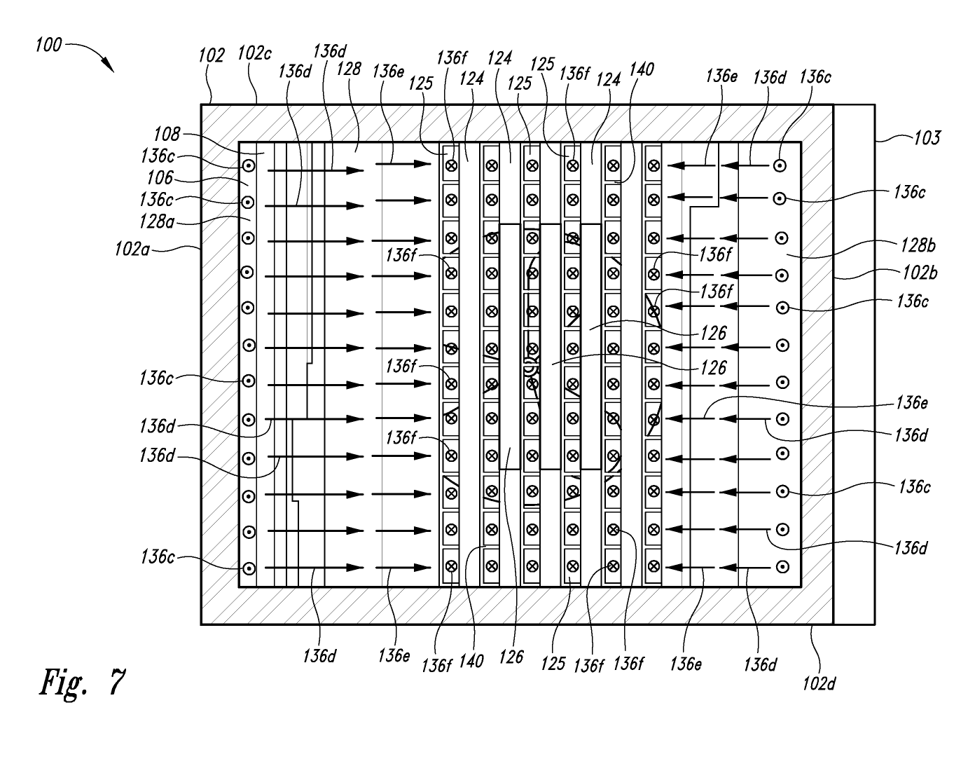

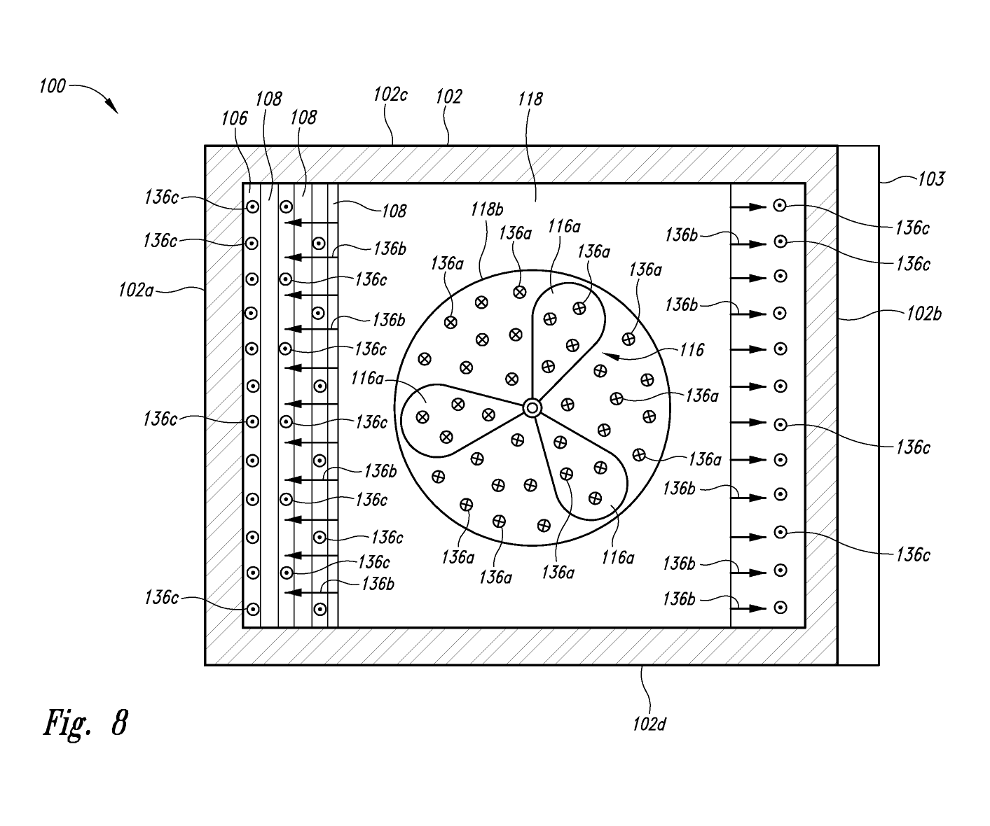

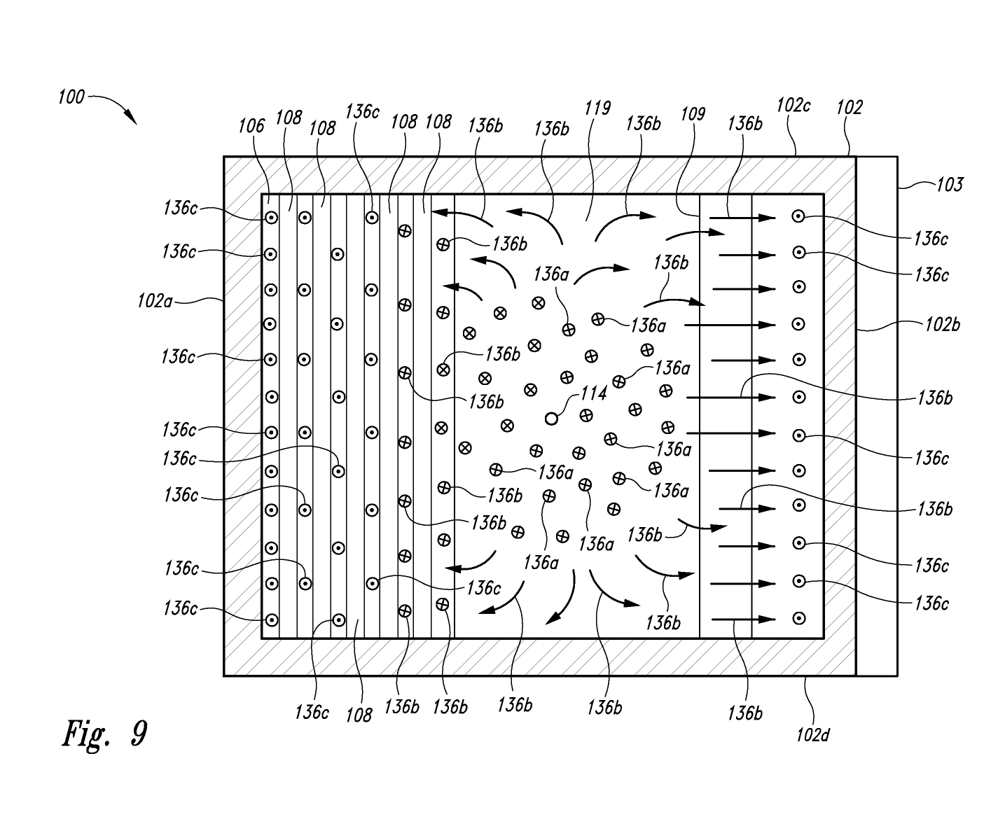

FIGS. 6-9 show airflow when the fan 110 is in the first mode of operation. As shown in FIGS. 6-9, the airflow 136c through the left air passageway 128a and the right air passageway 128b is substantially vertically undifferentiated. As shown in FIGS. 6-7, the airflow 136d from the left air passageway 128a and the right air passageway 128b into the product treatment area 122 is substantially vertically undifferentiated. As shown in FIGS. 6-7, the airflow 136e in the product treatment area 122 is substantially vertically undifferentiated so that product 105c is heat treated substantially the same regardless of its vertical elevation in the product treatment area. As shown in FIGS. 6-7, the airflow 136f approaching and passing into the louver assembly 120 is substantially vertically undifferentiated. As shown in FIGS. 8-9, the airflow 136b passing by the exchanger tubes 108 and the steam system 109 is substantially vertically undifferentiated so is treated substantially similarly regardless of vertical elevation in the air treatment area 119.

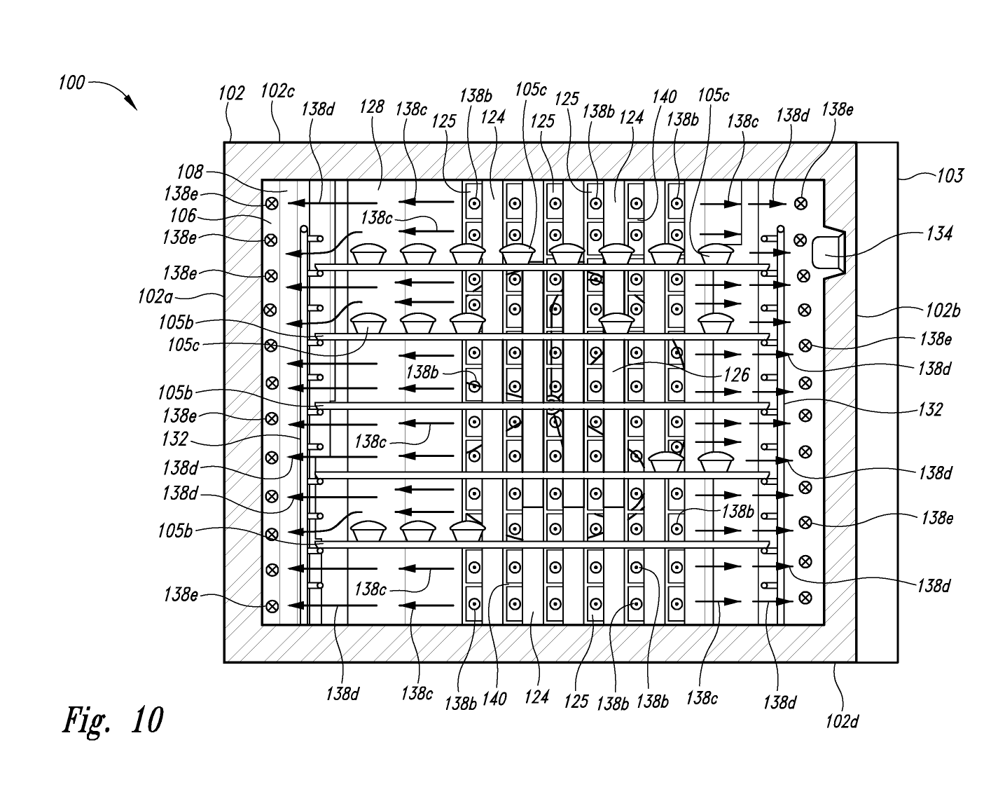

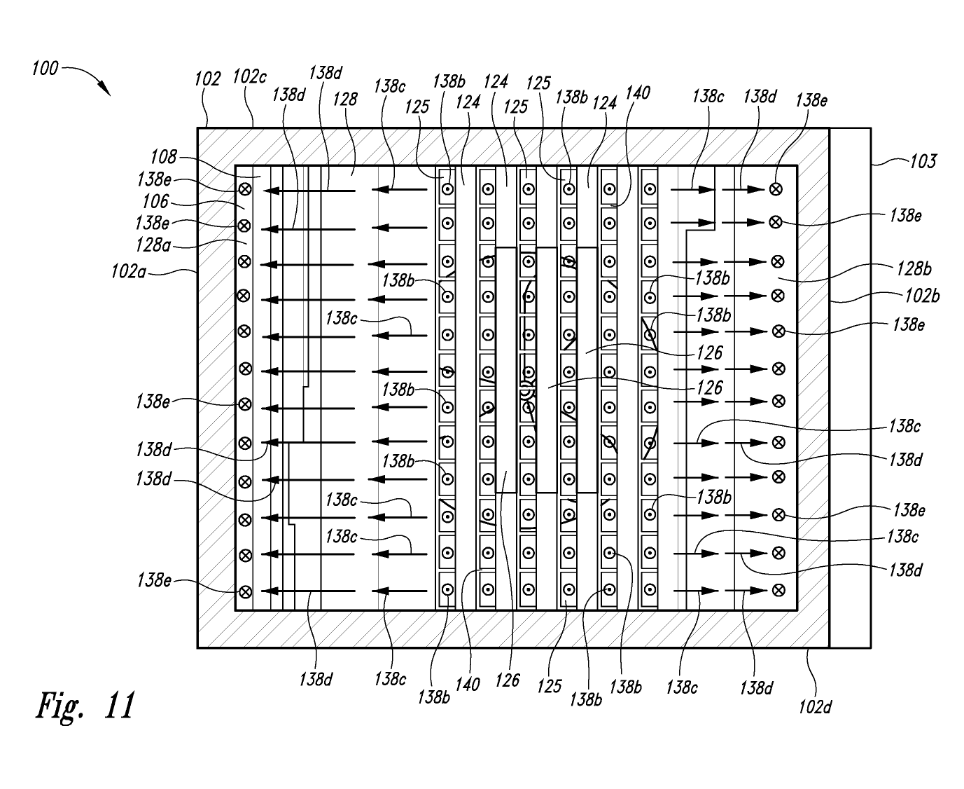

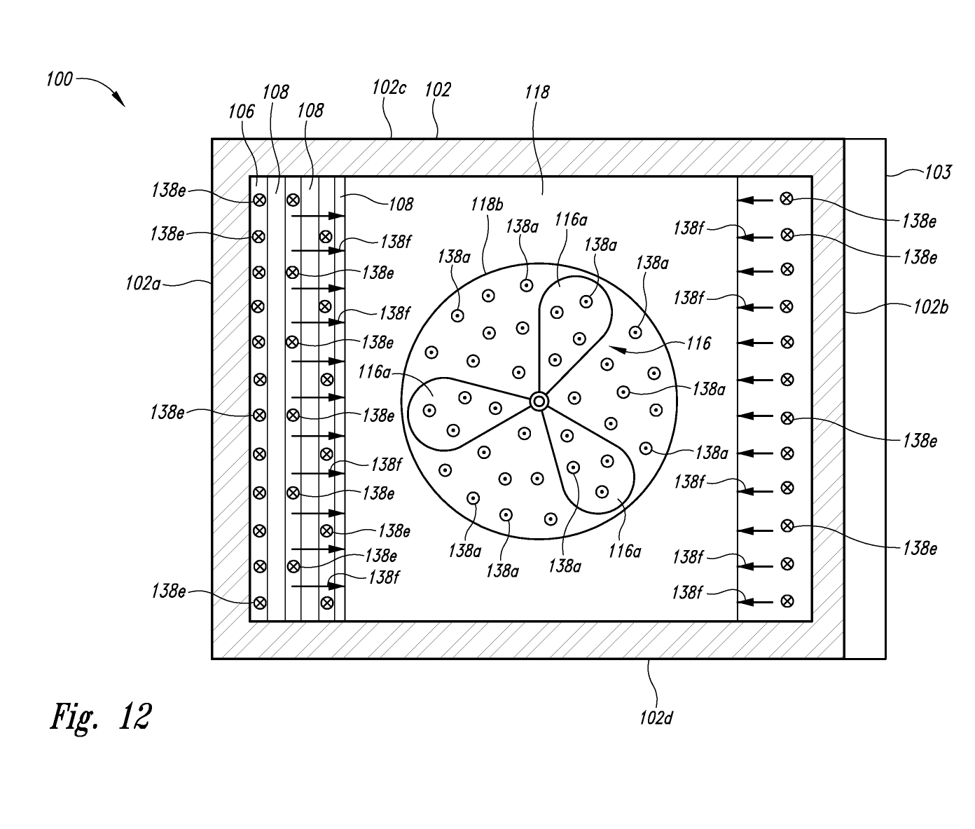

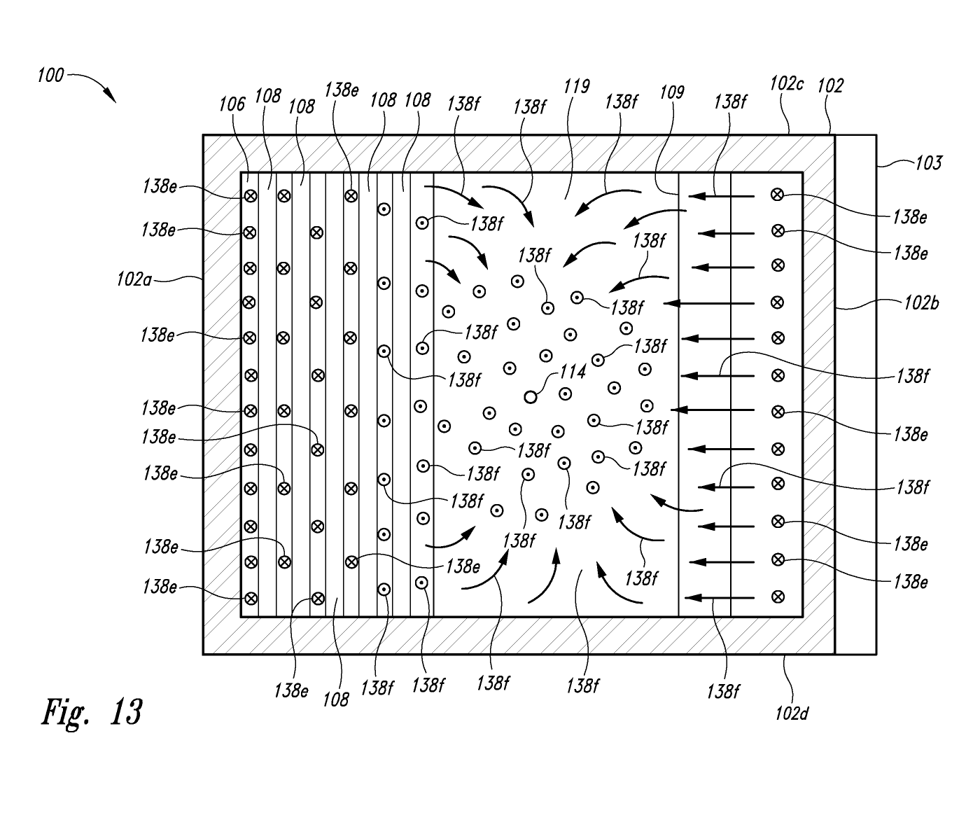

FIGS. 10-13 show airflow when the fan 110 is in the second mode of operation. As shown in FIGS. 10-13, the airflow 138e through the left air passageway 128a and the right air passageway 128b is substantially vertically undifferentiated. As shown in FIGS. 10-11, the airflow 138d into the left air passageway 128a and the right air passageway 128b from the product treatment area 122 is substantially vertically undifferentiated. As shown in FIGS. 10-11, the airflow 138c in the product treatment area 122 is substantially vertically undifferentiated so that product 105c is heat treated substantially the same regardless of its vertical elevation in the product treatment area. As shown in FIGS. 10-11, the airflow 138b leaving from the louver assembly 120 and passing into the product treatment area 122 is substantially vertically undifferentiated. As shown in FIGS. 12-13, the airflow 138f passing by the exchanger tubes 108 and the steam system 109 is substantially vertically undifferentiated so is treated substantially similarly regardless of vertical elevation in the air treatment area 119.

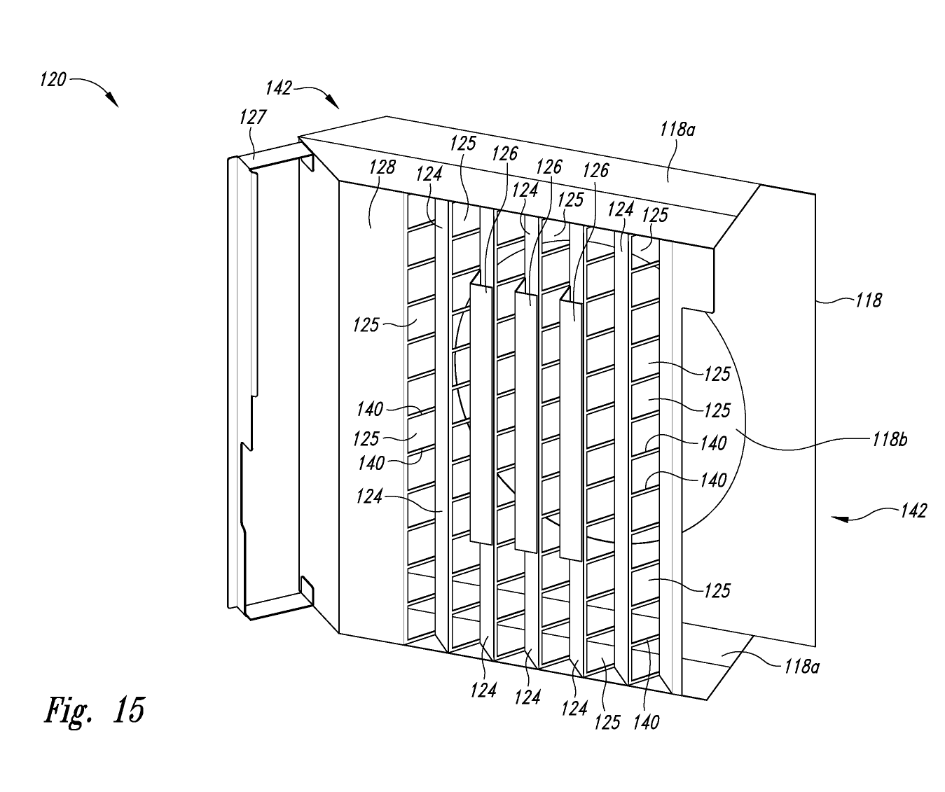

As shown in FIGS. 14-15, the shroud 118 has structural members 118a for coupling with the louver assembly 120. The shroud 118 has an opening 118b to receive and encompass the propeller 116 and is generally aligned with the center of the propeller. Diameter of the opening 118b is sized to regulate airflow volume and velocity as needed for airflow characteristics described herein. As assembled together, the shroud 118 and the louver assembly 120 provide large vertically oriented openings 142 to be used with maintaining desired airflow as described.

From the foregoing it will be appreciated that, although specific embodiments of the invention have been described herein for purposes of illustration, various modifications may be made without deviating from the spirit and scope of the invention. For instance in some implementations the steam system 109 may not be present while airflow will maintain its characteristics as discussed above. The air distribution panel could have other configurations than shown in louver assembly 120, such as horizontal slats, other shaped openings, or could otherwise distribute airflow according to objectives discussed. Furthermore, while the depicted implementation has the fan 110, the air distribution panel as the louver assembly 120, and the intermediate panel as the shroud 118 located toward the rear wall 102e, in other implementations, these components can be located toward the left side wall 102a or the right side wall 102b while still having similar results with airflow and even heat-treatment of product. Further, in some instances, the shape, hole pattern, and various baffles of the air distribution panel may be modified and still maintain an even heat-treatment of product, such as an even bake. Likewise, vertical openings at the sides of the air distribution panel may be modified and still maintain an even heat treatment such as an even bake. Accordingly, the invention is not limited except as by the appended claims.

The invention claimed is:

1. A system comprising:
a housing having a first wall, a second wall, a third wall, and a door;
an intermediate panel having a first end and a second end, the intermediate panel positioned a first distance from the first wall to bound an air treatment area therebetween, the first end spaced apart from a first selected of the second wall, the third wall, and the door to partially bound a first passageway, the second end spaced apart from a different second selected of the second wall, the third wall, and the door to partially bound a second passageway;
an air distribution panel having a first end and a second end, the air distribution panel positioned a second distance from the first wall, the second distance being greater than the first distance to bound a staging area between the air distribution panel and the intermediate panel, the first end spaced apart from the first selected wall to partially bound the first passageway, the second end spaced apart from the second selected to partially bound the second passageway, the air distribution panel positioned from a different third selected of the second wall, the third wall, and the door to bound a product treatment area therebetween, the product treatment area sized to contain product;
a fan with a propeller, the fan configured to alternatively spin the propeller in a first rotational direction and in an opposite second rotational direction, the propeller positioned to move air into the air treatment area when spinning the propeller in the first rotational direction and to move air into the staging area when spinning the propeller in the second rotation direction opposite the first rotational direction; and
a heating system configured to heat air while adjacent to a portion of the heating system, the portion being located in the air treatment area.

2. The system of claim 1 wherein the first wall is the rear wall opposingly positioned from the door.

3. The system of claim 1 wherein the air distribution panel is a louver assembly.

4. The system of claim 1 wherein the intermediate panel is a shroud including an opening size to encompass the propeller.

5. A system comprising:
a heating system;
a product treatment area;
an air treatment area having at least a portion of the heating system to heat air;
a staging area;
at least one passageway extending between the air treatment area and the product treatment area;
an air distribution panel positioned between the product treatment area and the staging area;
an intermediate panel positioned between the air treatment area and the staging area; and
a fan with a propeller, the fan configured to spin the propeller in a first rotational direction and a second opposite rotational direction, the fan positioned to move air into the air treatment area while the propeller is spinning in the first rotational direction thereby increasing pressure in the air treatment area compared with air pressure in the product treatment area, the fan positioned to move air into the staging area while the propeller is spinning in the second rotational direction thereby increasing pressure in the product treatment area compared with air pressure in the air treatment area.
